(19) United States
(12) Patent Application Publication
(10) Pub. No.: US 2022/0211925 A1
(43) Pub. Date: Jul. 7, 2022

(54) OXYGENATOR WITH WOUND FILTER MEMBRANE AND FLOW DIFFUSER

(71) Applicant: CardiacAssist, Inc., Pittsburgh, PA (US)

(72) Inventors: William Gesler, New Hudson, MI (US); Anthony McCoppin, Blawnox, PA (US); Michael J. Linehan, Pittsburgh, PA (US); Robert G. Svitek, Freeport, PA (US)

(73) Assignee: CardiacAssist, Inc., Pittsburgh, PA (US)

(21) Appl. No.: 17/706,304

(22) Filed: Mar. 28, 2022

Related U.S. Application Data
(63) Continuation of application No. PCT/US2020/055201, filed on Oct. 12, 2020.
(60) Provisional application No. 62/915,175, filed on Oct. 15, 2019.

(51) Int. Cl. A61M 1/16

(52) U.S. Cl. CPC A61M 1/1698; A61M 2206/14; B01D 2313/20; A61M 2206/18; A61M 2206/16

(57) ABSTRACT

A blood oxygenator has a housing with a first end opposite a second end and a sidewall extending between the first end and the second end along a longitudinal axis. The housing may define an interior chamber having a fluid inlet and a fluid outlet. The blood oxygenator further has a gas exchange medium positioned within the interior chamber. The gas exchange medium may have a plurality of hollow fibers rolled into a spiral shape. The blood oxygenator further has a flow diverter positioned within the interior chamber and configured for guiding fluid flow through the gas exchange medium.

### CROSS REFERENCE TO RELATED APPLICATIONS

This application is a continuation of International Application No. PCT/US2020/055201, filed Oct. 12, 2020, which claims the benefit of and priority to U.S. Provisional Patent Application Ser. No. 62/915,175, filed on Oct. 15, 2019, the disclosures of which are incorporated herein by reference.

### BACKGROUND OF THE DISCLOSURE

### Field of the Disclosure

The present disclosure is generally related to an apparatus that allows for the exchange of gases in a liquid sample. More specifically, the disclosure relates to a blood oxygenator having a wound filter membrane and a flow diffuser to allow external oxygen to be incorporated into a blood sample while carbon dioxide is removed from the blood sample.

### Description of Related Art

Blood oxygenators are commonly used to accomplish the gas exchange functions normally performed by the lungs. Conventional blood oxygenators contain a gas exchange medium, such as a filter membrane made from hollow fibers, across which blood is flowed. The filter membrane is connected to an oxygen supply such that oxygen is diffused from the filter membrane into the blood and carbon dioxide is removed from the blood into the filter membrane.

Conventional oxygenators are commonly used in medical situations when a patient's lungs are temporarily disabled and/or incapable of performing their normal function. In some medical situations, blood oxygenators are used as a temporary gas exchange member to substitute or supplement the lung function during, for example, open heart surgery. During such procedures, vital functions of the circulatory system are assumed by an extracorporeal bypass circuit where a pump sends the patient's blood through a blood oxygenator to deliver oxygen to the patient. In other medical situations, a patient may have an indwelling catheter connected to a pump to deliver blood to a blood oxygenator. In these applications, the oxygenator can be used for an indefinite term.

Membrane blood oxygenators transfer oxygen into the blood as it flows over a bundle of hollow fiber membranes. The liquid side boundary layer is the limiting factor in transferring oxygen. Increasing the mixing of blood around the hollow fiber membranes decreases the liquid side boundary layer thickness and increases the exchange of oxygen. A solution for existing blood oxygenators is to increase the amount of fiber membrane surface area or to increase the mixing of blood around the fibers through impellers or rotation of the fibers. However, increasing the amount of fiber surface area increases the foreign surface to blood contact area, which can lead to adverse events such as thrombosis or platelet activation. Additionally, implementing active mixing technologies adds complication to the manufacturing and design of the oxygenator.

There is a need in the art for a blood oxygenator that is suitable for use for an indefinite term to provide gas exchange function without imposing a significant load onto the patient's heart. It would be further desirable to have a blood oxygenator having an increased gas exchange efficiency and a smaller size compared to conventional blood oxygenators.

### SUMMARY OF THE DISCLOSURE

In some examples or aspects of the present disclosure, an improved blood oxygenator is provided for use for an indefinite term to provide gas exchange function without imposing a significant load onto the patient's heart. The improved blood oxygenator has an increased gas exchange efficiency and a small size.

In some examples or aspects of the present disclosure, a blood oxygenator may have a housing with a first end opposite a second end and a sidewall extending between the first end and the second end along a longitudinal axis. The housing may define an interior chamber having a fluid inlet and a fluid outlet. The blood oxygenator may have a gas exchange medium positioned within the interior chamber. The gas exchange medium may have a plurality of hollow fibers rolled into a spiral shape. The blood oxygenator may have a flow diverter positioned within the interior chamber and configured for guiding fluid flow through the gas exchange medium.

In other examples or aspects of the present disclosure, the flow diverter may have a fixed end connected to a central portion of the housing and a free end extending from the first end along the longitudinal axis. The flow diverter may have a spiral shape between the fixed end and the free end. A diameter of the flow diverter may increase or decrease between the fixed end and the free end. The flow diverter may extend along 25% to 100% of a longitudinal length of the gas exchange medium.

In other examples or aspects of the present disclosure, the flow diverter may have one or more annular sleeves extending longitudinally through the gas exchange medium. The one or more sleeves may be offset longitudinally relative to each other to define a tortuous fluid path therebetween. The one or more sleeves may be arranged concentrically relative to the longitudinal axis. The flow diverter may be a baffle positioned between a first section of the gas exchange medium and a second section of the gas exchange medium. The baffle may be configured to permit at least a portion of the fluid flow to pass through the baffle in a radial direction. The flow diverter may be a screen having a plurality of openings, pores, or slots. A size of the openings, pores, or slots may increase or decrease between the first end and the second end of the housing. The one or more sleeves, baffle, or screen may include a combination of regions with openings, pores, and/or slots.

In other examples or aspects of the present disclosure, the flow diverter may include at least one first ring and at least one second ring arranged in an alternating manner Each first ring may be a solid plate and each second ring may be an annular plate.

In other examples or aspects of the present disclosure, the flow diverter may be an inflatable balloon positioned in a central portion of the interior chamber. The inflatable balloon may be in fluid communication with a pump via a fluid line, and wherein the pump is configured for selectively inflating or deflating the inflatable balloon via the fluid line.

Various other aspects of the present disclosure are recited in one or more of the following clauses:

Clause 1: A blood oxygenator comprising: a housing having a first end opposite a second end with a sidewall extending between the first end and the second end along a longitudinal axis, the housing defining an interior chamber having a fluid inlet and a fluid outlet; a gas exchange medium positioned within the interior chamber, the gas exchange medium having a plurality of hollow fibers rolled into a spiral shape; and a flow diverter positioned within the interior chamber and configured for guiding fluid flow through the gas exchange medium.

Clause 2. The blood oxygenator of clause 1, wherein the flow diverter has a fixed end connected to a central portion of the housing and a free end extending from the first end along the longitudinal axis, and wherein the flow diverter has a spiral shape between the fixed end and the free end.

Clause 3. The blood oxygenator of clause 2, wherein a diameter of the flow diverter increases or decreases between the fixed end and the free end.

Clause 4. The blood oxygenator of any of clauses 1-3, wherein the flow diverter extends along 25% to 100% of a longitudinal length of the gas exchange medium.

Clause 5. The blood oxygenator of any of clauses 1-4, wherein the flow diverter has one or more annular sleeves extending longitudinally through the gas exchange medium.

Clause 6. The blood oxygenator of clause 5, wherein the one or more sleeves are offset longitudinally relative to each other to define a tortuous fluid path therebetween.

Clause 7. The blood oxygenator of clause 5 or 6, wherein the one or more sleeves are arranged concentrically relative to the longitudinal axis.

Clause 8. The blood oxygenator of clause 1, wherein the flow diverter is a baffle positioned between a first section of the gas exchange medium and a second section of the gas exchange medium and wherein the baffle is configured to permit at least a portion of the fluid flow to pass through the baffle in a radial direction.

Clause 9. The blood oxygenator of clause 1, wherein the flow diverter is a screen having a plurality of openings, pores, or slots.

Clause 10. The blood oxygenator of clause 9, wherein a size of the openings, pores, or slots increases or decreases between the first end and the second end of the housing.

Clause 11. The blood oxygenator of any one of clauses 5-10, wherein the one or more sleeves, baffle, or screen may include a combination of regions with openings, pores, and/or slots.

Clause 12. The blood oxygenator of clause 1, wherein the flow diverter includes at least one first ring and at least one second ring arranged in an alternating manner.

Clause 13. The blood oxygenator of clause 12, wherein each first ring is a solid plate and each second ring is an annular plate.

Clause 14. The blood oxygenator of clause 1, wherein the flow diverter is an inflatable balloon positioned in a central portion of the interior chamber.

Clause 15. The blood oxygenator of clause 14, wherein the inflatable balloon is in fluid communication with a pump via a fluid line, and wherein the pump is configured for selectively inflating or deflating the inflatable balloon via the fluid line.

Further details and advantages of the various examples or aspects described in detail herein will become clear upon reviewing the following detailed description of the various examples in conjunction with the accompanying drawing figures.

### BRIEF DESCRIPTION OF THE DRAWINGS

FIG. 1 is a cross-sectional view of a blood oxygenator in accordance with one example or aspect of the present disclosure;

FIG. 2A is a cross-sectional view of a blood oxygenator in accordance with another example or aspect of the present disclosure;

FIG. 2B is a cross-sectional view of a blood oxygenator in accordance with another example or aspect of the present disclosure;

FIG. 3 is a cross-sectional view of a blood oxygenator in accordance with another example or aspect of the present disclosure;

FIG. 4 is a cross-sectional view of a blood oxygenator in accordance with another example or aspect of the present disclosure;

FIG. 5 is a side view of an insert for use with the blood oxygenator of FIG. 3 or FIG. 4; and

FIG. 6 is a cross-sectional view of a blood oxygenator in accordance with another example or aspect of the present disclosure.

### DETAILED DESCRIPTION OF THE INVENTION

The illustrations generally show preferred and non-limiting examples or aspects of the apparatus and methods of the present disclosure. While the description presents various aspects of the apparatus, it should not be interpreted in any way as limiting the disclosure. Furthermore, modifications, concepts, and applications of the disclosure's aspects are to be interpreted by those skilled in the art as being encompassed, but not limited to, the illustrations and descriptions herein.

The following description is provided to enable those skilled in the art to make and use the described examples contemplated for carrying out the disclosure. Various modifications, equivalents, variations, and alternatives, however, will remain readily apparent to those skilled in the art. Any and all such modifications, variations, equivalents, and alternatives are intended to fall within the spirit and scope of the present disclosure.

For purposes of the description hereinafter, the terms “upper”, “lower”, “right”, “left”, “vertical”, “horizontal”, “top”, “bottom”, “lateral”, “longitudinal”, and derivatives thereof shall relate to the disclosure as it is oriented in the drawing figures.

As used herein, the terms “parallel” or “substantially parallel” mean a relative angle as between two objects (if extended to theoretical intersection), such as elongated objects and including reference lines, that is from 0° to 5°, or from 0° to 3°, or from 0° to 2°, or from 0° to 1°, or from 0° to 0.5°, or from 0° to 0.25°, or from 0° to 0.1°, inclusive of the recited values.

As used herein, the term “perpendicular” or “substantially perpendicular” mean a relative angle as between two objects (if extended to theoretical intersection), such as elongated objects and including reference lines, that is from 85° to 90°, or from 87° to 90°, or from 88° to 90°, or from 89° to 90°, or from 89.5° to 90°, or from 89.75° to 90°, or from 89.9° to 90°, inclusive of the recited values.

It is to be understood, however, that the disclosure may assume alternative variations and step sequences, except where expressly specified to the contrary. It is also to be understood that the specific devices and processes illustrated in the attached drawings, and described in the following specification, are simply exemplary aspects of the disclosure. Hence, specific dimensions and other physical characteristics related to the examples disclosed herein are not to be considered as limiting.

It should be understood that any numerical range recited herein is intended to include all sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of equal to or less than 10.

In this application, the use of the singular includes the plural and plural encompasses singular, unless specifically stated otherwise. In addition, in this application, the use of “or” means “and/or” unless specifically stated otherwise, even though “and/or” may be explicitly used in certain instances. Further, in this application, the use of “a” or “an” means “at least one” unless specifically stated otherwise.

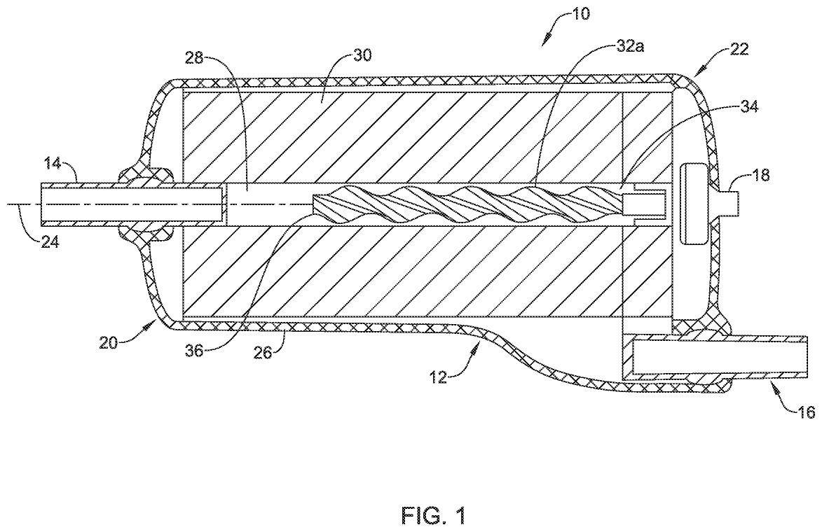

Referring to FIG. 1, a blood oxygenator 10 is shown in accordance with one example or aspect of the present disclosure. The blood oxygenator 10 may be suitable for use in an extracorporeal membrane oxygenation (ECMO) system. The blood oxygenator 10 has a housing 12 having a liquid inlet 14, a liquid outlet 16, a gas inlet 18, and a gas outlet (not shown). The housing 12 has a first end 20 opposite a second end 22 along a longitudinal axis 24. A sidewall 26 extends between the first end 20 and the second end 22 and encloses an interior chamber 28 that provides the space in which gas exchange functions are performed. A gas exchange medium 30 is positioned within the interior chamber 28.

With continued reference to FIG. 1, the housing 12 may have a circular or oval cross-sectional shape and may be made from a rigid material, such as a biocompatible plastic. The plastic may be transparent, translucent, or opaque.

With continued reference to FIG. 1, the liquid inlet 14, the liquid outlet 16, the gas inlet 18, and the gas outlet are in fluid communication with the interior chamber 28. In some examples or aspects, the liquid outlet 16, the gas inlet 18, and the gas outlet may have features that facilitate connection to another device, such as tubing or the like. Such features can include barbs, clamps, and/or any other suitable connection feature.

With continued reference to FIG. 1, the gas exchange medium 30 is disposed within the interior chamber 28 and is configured for diffusing a gas flowing therethrough into the liquid flowing around the gas exchange medium 30. In some examples or aspects, the gas exchange medium 30 has a plurality of individual hollow fibers, such as those discussed in U.S. Pat. No. 6,682,698. The fibers are configured to carry a gas, such as oxygen, in such a manner that allows the gas to be taken up by a liquid, such as blood, flowing around the fibers, and to absorb any other gas given off by the liquid, such as carbon dioxide. The gas exchange medium 30 provides the required surface area for the gas exchange to occur. In some examples or aspects, the gas exchange medium 30 may be a woven fiber mat that is rolled into a spiral shape about the longitudinal axis 24. In this manner, the gas exchange medium 30 may have an outer diameter than is configured to fit within an inner diameter of the housing 12 and an inner diameter inside of which a flow diverter is received, as discussed herein. Alternatively, the gas exchange medium 30 can be any other gas exchange medium known in the art. A potting material (not shown) may be used to seal the inlet and outlet sides of the hollow fibers of the gas exchange medium 30 in order to prevent direct mixing of the gas flowing through the fibers with the liquid flowing around the fibers.

During operation, blood enters the oxygenator 10 through the liquid inlet 14 along an axial path extending along the longitudinal axis 24 from the first end 20 toward the second end 22. The blood must be radially diverted so that it can pass through the gas exchange medium 30. Various devices for radially diverting the flow of blood within the interior chamber 28 and promoting mixing flow of the blood between the fibers of the gas exchange medium 30 are disclosed herein with reference to FIGS. 1-6.

With continued reference to FIG. 1, a flow diverter 32a is shown in accordance with one example or aspect of the present disclosure. The flow diverter 32a is positioned within the interior chamber 28 at a central portion of the housing 12 and in a central opening within the gas exchange medium 30. The flow diverter 32a is configured to divert the blood flowing along an axial path in a radial direction toward the gas exchange medium 30. The flow diverter 32a has a first, fixed end 34 fixedly connected to the second end 22 of the housing 12, and a second, free end 36 opposite the first end 34. The second, free end 36 is directed toward the liquid inlet 14 such that liquid entering the interior chamber 28 through the liquid inlet 14 is directed toward the flow diverter 32a. The flow diverter 32a desirably extends along 25% to 100% of the length of the gas exchange medium 30. In some instances, the flow diverter 32a extends along 50% or more, or 60% or more, or 75% or more of the length of the gas exchange medium 30.

With continued reference to FIG. 1, the flow diverter 32a has a spiral shape having 0.5 to 10 twists per inch in a direction about the longitudinal axis 24 between the first end 34 and the second end 36. In some instances, the flow diverter 32a has a spiral shape having 0.5 to 5 twists per inch, 5 to 10 twists per inch, 2 to 8 twist per inch, or 3 to 10 twists per inch in a direction about the longitudinal axis 24. In some examples or aspects, a diameter of the flow diverter 32a may be uniform along its length. In other examples or aspects, the diameter of the flow diverter 32a may increase or decrease in a direction from the first end 34 toward the second end 36. The flow diverter 32a may have a diameter that is 90-95% of the inner diameter of the gas exchange medium 30. In some instances, the flow diverter 32a may have a diameter that is 80% or more, 85% or more, or 90% or more of the inner diameter of the gas exchange medium 30. The spiral shape of the flow diverter 32a imparts a spiral flow to the blood flowing in through the liquid inlet 14. This results in an even radial distribution of flow across the gas exchange medium 30. Additionally, gas exchange is improved due to a more tortuous fluid path compared to oxygenators without the flow diverter 32a.

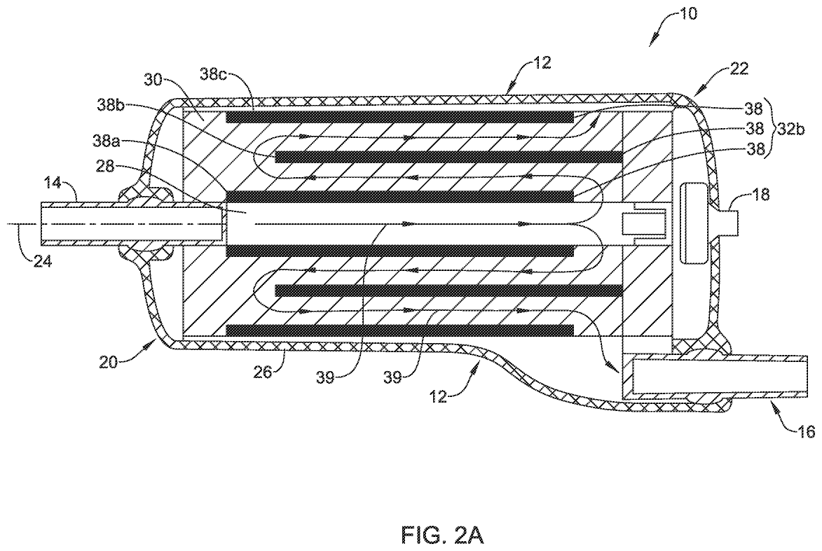

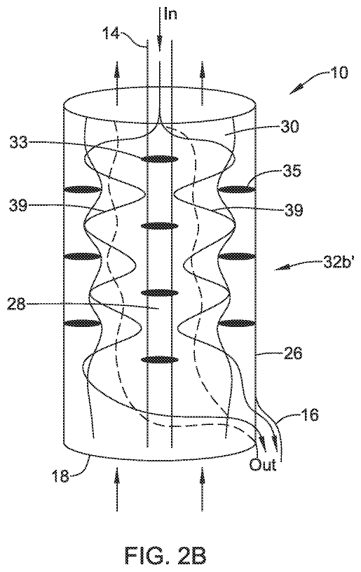

With reference to FIG. 2A, a blood oxygenator 10 having a flow diverter 32b is shown in accordance with another example or aspect of the present disclosure. The flow diverter 32b has one or more annular sleeves 38 positioned within the interior chamber 28. In some examples or aspects, the one or more sleeves 38 may be positioned within the gas exchange medium 30. The one or more sleeves 38 are arranged concentrically relative to the longitudinal axis 24, with a first sleeve 38a positioned closest to the longitudinal axis 24 and the remaining sleeves 38b-38c positioned radially outward relative to the first sleeve 38a. The sleeves 38a-38c are axially offset from one another such that a tortuous path 39 is defined between the sleeves 38a-38c and through the gas exchange medium 30. For example, the sleeves 38a-38c may be arranged such that the end of one or more of the sleeves 38a-38c closest to the first end 20 is closer to the first end 20 than one or more of the other sleeves 38a-38c and/or the end of one or more of the sleeves 38a-38c closest to the second end 22 is closer to the second end 22 than one or more of the other sleeves 38a-38c. In this manner, blood flowing around the fibers of the gas exchange medium 30 must take the tortuous path 39 around the sleeves 38a-38c when flowing from the liquid inlet 14 to the liquid outlet 16.

With continued reference to FIG. 2A, the sleeves 38a-38c of the flow diverter 32b may extend along 50% to 90% of the length of the gas exchange medium 30. In some instances, the sleeves 38a-38c extend along 50% or more, or 60% or more, or 75% or more of the length of the gas exchange medium 30. In some examples or aspects, at least one of the sleeves 38a-38c may be made from a solid material such that the sleeve is configured to block fluid flow in a radial direction and promote fluid flow in an axial direction along the longitudinal axis 24. In other examples, at least one of the sleeves 38a-38c may permit at least a portion of the fluid flow to pass through the sleeve in a radial direction. For example, the at least one of the sleeves 38a-38c may be made from a porous material, or have one or more slots or openings, as discussed herein. The sleeves 38a-38c are configured to promote even radial distribution of fluid flow and lower the pressure drop across the gas exchange medium 30. The cross-sectional area of the oxygenator 10 having the flow diverter 32b is reduced in the regions where the sleeves 38a-38c are present, thereby increasing the velocity of the blood flowing through the tortuous path 39, which in turn increases the gas exchange rate.

With reference to FIG. 2B, a blood oxygenator 10 having a flow diverter 32b′ is shown in accordance with another example or aspect of the present disclosure. The flow diverter 32b′ has at least one first ring 33 and at least one second ring 35. The at least one first ring 33 and the at least one second ring 35 may be arranged in an alternating manner wherein no two first rings 33 or second rings 35 are placed adjacent to each other. Thus, a first ring 33 may be positioned longitudinally between two second rings 35 and/or a second ring 35 may be positioned longitudinal between two first rings 33. Each first ring 33 may be a solid plate that is positioned within an inner diameter of the gas exchange medium 30. Each second ring 35 may be an annular plate that is positioned outside an outer diameter of the gas exchange medium. Thus, the inner diameter of the second rings 35 may be greater than the outer diameter of the first rings 33. The first and second rings 33, 35 are spaced apart from each other in a direction along the longitudinal axis 24 such that blood must follow a tortuous path 39 as it moves from the liquid inlet 14 to the liquid outlet 16.

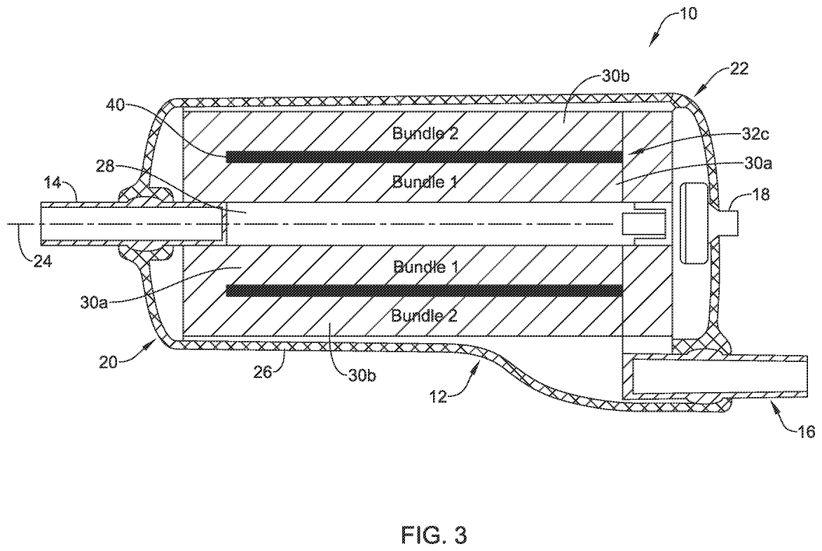

With reference to FIG. 3, a blood oxygenator 10 having a flow diverter 32c is shown in accordance with another example or aspect of the present disclosure. The flow diverter 32c is configured as a baffle 40 positioned between a pair of gas exchange mediums 30a, 30b. In some examples or aspects, a plurality of baffles 40 may be provided to separate a plurality of gas exchange mediums. Each gas exchange medium 30a, 30b may be a woven fiber mat that is rolled into a spiral shape about the longitudinal axis 24. The gas exchange mediums 30a, 30b are arranged concentrically relative to the longitudinal axis 24, with a first gas exchange medium 30a positioned closest to the longitudinal axis 24 and the second gas exchange medium 30b positioned radially outward relative to the first gas exchange medium 30a.

With continued reference to FIG. 3, the baffle 40 is positioned between the gas exchange mediums 30a, 30b. In some examples or aspects, the baffle 40 extends along the entire longitudinal length of the of the gas exchange mediums 30a, 30b. In other examples or aspects, the baffle 40 extends along a portion of the longitudinal length of the gas exchange mediums 30a, 30b, as shown in FIG. 3. In some instances, the baffle 40 extends along 50% or more, or 60% or more, or 75% or more of the length of the gas exchange mediums 30a, 30b. The baffle 40 is configured to permit at least a portion of the fluid flow to pass through the baffle 40 in a radial direction. For example, the baffle 40 may be made from a porous material, or have one or more slots or openings, as discussed herein.

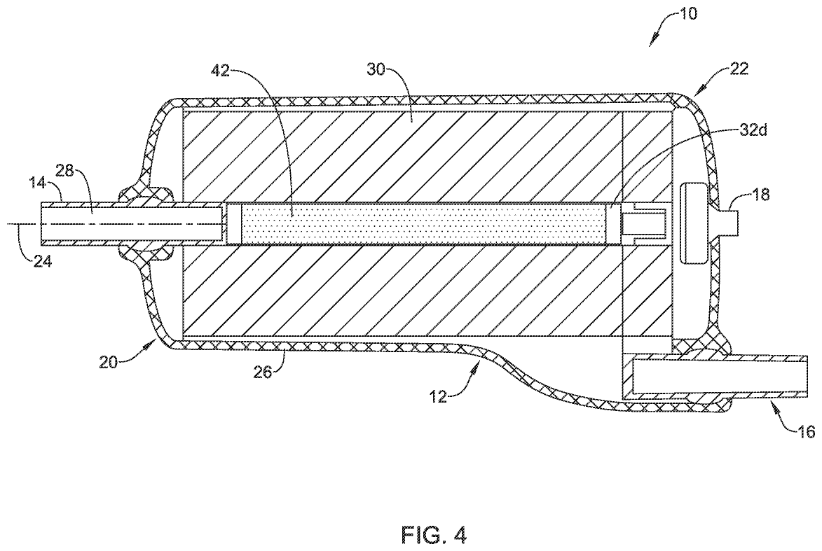

With reference to FIG. 4, a blood oxygenator 10 having a flow diverter 32d is shown in accordance with another example or aspect of the present disclosure. The flow diverter 32d is configured as a screen 42 having a tubular shape and is positioned at a radially inward position of the gas exchange medium 30. The flow diverter 32d may extend along 25% to 100% of the length of the gas exchange medium 30. In some instances, the flow diverter 32d may extend along 50% or more, or 60% or more, or 75% or more of the length of the gas exchange medium 30. The flow diverter 32d is configured to permit at least a portion of the fluid flow to pass therethrough in a radial direction. For example, the flow diverter 32d may be made from a porous material, or have one or more slots or openings, as discussed herein. In some examples or aspects, the size or flow area of the openings through which fluid may flow through the flow diverter 32d may vary in a direction along the longitudinal axis 24. For example, the size of the openings, pores, or slots may vary (e.g., increase or decrease) between the first end 20 and the second end 22 of the housing 12.

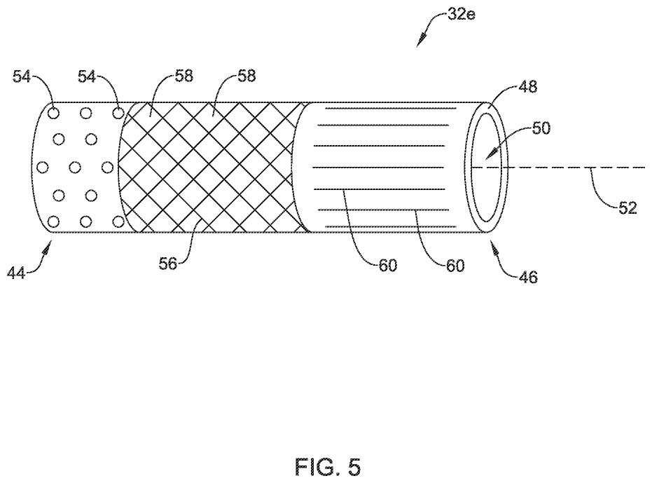

With reference to FIG. 5, a flow diverter 32e is shown in accordance with another example or aspect of the present disclosure. The flow diverter 32e may be used as one of the annular sleeves 38 shown in FIG. 2A, the baffle 40 shown in FIG. 3, or the screen 42 shown in FIG. 4. The flow diverter 32e has an annular shape having a first end 44, a second end 46, and sidewall 48 defining a central opening 50 between the first end 44 and the second end 46 along a central axis 52. In some examples or aspects, the flow diverter 32e has a plurality of openings 54 extending through the sidewall 48. Each of the plurality of openings 54 is configured to permit fluid to flow therethrough. The openings 54 may have a substantially circular shape with a uniform or a non-uniform diameter in a direction along the longitudinal axis 24. In some examples or aspects, the size of the openings 54 may increase from the first end 44 to the second end 46.

In further examples or aspects, the flow diverter 32e may be made from a mesh 56 defining a plurality of pores 58 configured to permit fluid to flow therethrough. The pores 58 may have a substantially quadrilateral shape. In some examples or aspects, the size of the pores 58 may increase from the first end 44 to the second end 46.

In further examples or aspects, the flow diverter 32e may have a plurality of slots 60 configured to permit fluid to flow therethrough. The slots 60 may have an elongated shape. In some examples or aspects, the size (i.e., width) of the slots 60 may increase from the first end 44 to the second end 46.

In further examples or aspects, the flow diverter 32e may include a combination of regions with openings 54, mesh 56 with pores 58, and/or slots 60.

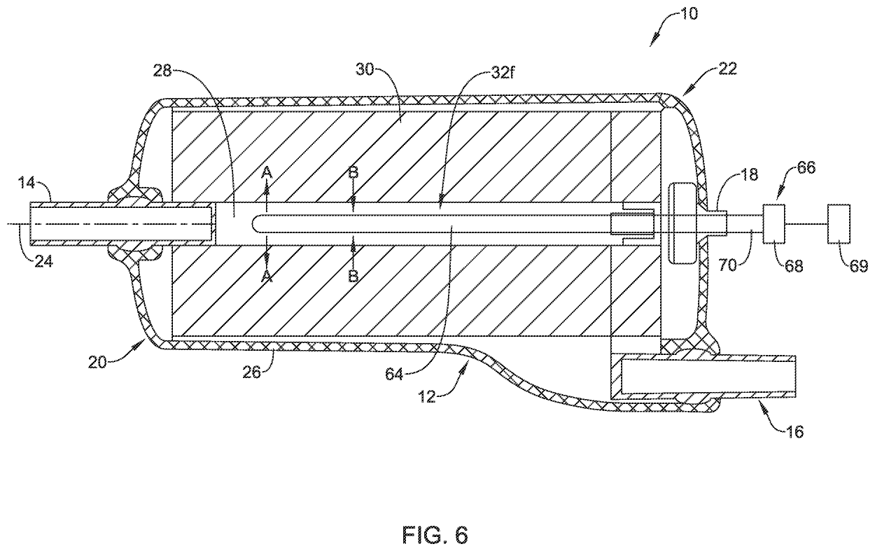

With reference to FIG. 6, the blood oxygenator 10 is shown in accordance with another example or aspect. The blood oxygenator 10 has a flow diverter 32f in the form of an inflatable balloon 64 positioned within the interior chamber 28. In some examples or aspects, the balloon 64 is positioned in a central portion of the interior chamber 28 and in a central hollow portion of the gas exchange medium 30. The balloon 64 is connected to a fluid source 66 that is configured for selectively inflating or deflating the balloon 64. In some examples or aspects, the fluid source 66 may be a fluid pump 68 that is in fluid communication with the balloon 64 via a fluid line 70. In various examples or aspects, the balloon 64 may be inflated and deflated via hydraulic, pneumatic, mechanical, electrical, or electromechanical devices, including any combinations thereof. A controller 69 may be operatively connected to the fluid pump 68 for controlling the flow of fluid to and from the balloon 64. Using the pump 68, pressurized fluid is delivered to the balloon 64 via the fluid line 70 to inflate the balloon 64 in a direction of arrows A. Conversely, by depressurizing the pump 68 or reversing its operating direction, the balloon 64 can be deflated in a direction of arrows B.

Inflation of the balloon 64 is configured to force the fluid within the interior chamber 28 to flow radially outward through the fibers of the gas exchange medium 30. In some examples or aspects, the balloon 64 may be expanded such that an outer diameter of the balloon 64 is the same as the inner diameter of the gas exchange medium 30 to force any fluid present in the space between the balloon 64 and the gas exchange medium 30 into the space between individual fibers of the gas exchange medium 30. Deflation of the balloon 64 allows additional fluid to enter the interior chamber 28 so that the fluid can be forced radially outward with the subsequent inflation of the balloon 64.

The controller 69 controls operation of the pump 68 to selectively inflate and deflate the balloon 64. In some examples or aspects, the controller 69 can be configured to operate the pump 68 in a pulsatile manner to selectively inflate and deflate the balloon 64 according to a pre-defined pressure profile. In other examples or aspects, the controller 69 can be configured to operate the pump 68 to selectively inflate and deflate the balloon 64 based on input from at least one sensor that measures a physiological characteristic of a patient. For example, the controller 69 can operate the pump 68 to inflate and deflate the balloon 64 based on input received from a heart rate sensor. Thus, inflation/deflation of the balloon 64 may be coordinated with the patient's heart rate.

The present disclosure also provides a method of operating a blood oxygenator. The method includes introducing blood into the interior chamber 28 through the liquid inlet 14 along an axial path in a direction of the longitudinal axis 24. The blood is radially diverted toward the outer portion of the interior chamber such that the blood passes around the fibers of the gas exchange medium 30. As the blood flows around the fibers of the gas exchange medium 30, gas exchange takes place between the blood and the gas flowing through the fibers of the gas exchange medium 30. In order to facilitate the gas exchange, the method further includes introducing a gas, such as oxygen or air, into the gas inlet 18 such that the gas passes through the gas exchange medium 30 and exits through the gas outlet. Oxygenated blood is directed through the liquid outlet 16. Radial diverting of the blood may be facilitated using one or more of the diverter, the baffle, the separator screen, or the inflatable balloon described herein.

While examples or aspects of an improved blood oxygenator are provided in the foregoing description, those skilled in the art may make modifications and alterations to these examples or aspects without departing from the scope and spirit of the disclosure. Accordingly, the foregoing description is intended to be illustrative rather than restrictive. The disclosure described hereinabove is defined by the appended claims, and all changes to the disclosure that fall within the meaning and the range of equivalency of the claims are to be embraced within their scope.

We claim:

1. A blood oxygenator comprising:
a housing having a first end opposite a second end with a sidewall extending between the first end and the second end along a longitudinal axis, the housing defining an interior chamber having a fluid inlet and a fluid outlet;
a gas exchange medium positioned within the interior chamber, the gas exchange medium having a plurality of hollow fibers rolled into a spiral shape; and
a flow diverter positioned within the interior chamber and configured for guiding fluid flow through the gas exchange medium.

2. The blood oxygenator of claim 1, wherein the flow diverter has a fixed end connected to a central portion of the housing and a free end extending from the first end along the longitudinal axis, and wherein the flow diverter has a spiral shape between the fixed end and the free end.

3. The blood oxygenator of claim 2, wherein a diameter of the flow diverter increases or decreases between the fixed end and the free end.

4. The blood oxygenator of claim 1, wherein the flow diverter extends along 25% to 100% of a longitudinal length of the gas exchange medium.

5. The blood oxygenator of claim 1, wherein the flow diverter has one or more annular sleeves extending longitudinally through the gas exchange medium.

6. The blood oxygenator of claim 5, wherein the one or more sleeves are offset longitudinally relative to each other to define a tortuous fluid path therebetween.

7. The blood oxygenator of claim 5, wherein the one or more sleeves are arranged concentrically relative to the longitudinal axis.

8. The blood oxygenator of claim 5, wherein the one or more sleeves includes a combination of regions with openings, pores, and/or slots.

9. The blood oxygenator of claim 1, wherein the flow diverter is a baffle positioned between a first section of the gas exchange medium and a second section of the gas exchange medium and wherein the baffle is configured to permit at least a portion of the fluid flow to pass through the baffle in a radial direction.

10. The blood oxygenator of claim 9, wherein the baffle includes a combination of regions with openings, pores, and/or slots.

11. The blood oxygenator of claim 1, wherein the flow diverter is a screen having a plurality of openings, pores, or slots.

12. The blood oxygenator of claim 11, wherein a size of the openings, pores, or slots increases or decreases between the first end and the second end of the housing.

13. The blood oxygenator of claim 12, wherein the screen includes a combination of regions with openings, pores, and/or slots.

14. The blood oxygenator of claim 1, wherein the flow diverter includes at least one first ring and at least one second ring arranged in an alternating manner.

15. The blood oxygenator of claim 14, wherein each first ring is a solid plate and each second ring is an annular plate.

16. The blood oxygenator of claim 1, wherein the flow diverter is an inflatable balloon positioned in a central portion of the interior chamber.

17. The blood oxygenator of claim 16, wherein the inflatable balloon is in fluid communication with a pump via a fluid line, and wherein the pump is configured for selectively inflating or deflating the inflatable balloon via the fluid line.
